(19) United States
(12) Patent Application Publication
(10) Pub. No.: US 2022/0261916 A1
(43) Pub. Date: Aug. 18, 2022

(54) CLOUD-BASED SYSTEM AND METHOD FOR CLAIMING INSURANCE FOR OUT-OF-POCKET MEDICAL EXPENSES

(71) Applicant: LEMONHEALTHCARE, Seoul (KR)

(72) Inventor: Byungjin HONG, Seoul (KR)

(73) Assignee: LEMONHEALTHCARE, Seoul (KR)

(21) Appl. No.: 17/626,251

(22) PCT Filed: Jul. 9, 2020
(86) PCT No.: PCT/KR2020/009058

§ 371 Date: Jan. 11, 2022

(30) Foreign Application Priority Data
Jul. 11, 2019 (KR) 10-2019-0083785

(51) Int. Cl. G06Q 40/08; G16H 10/60; H04L 9/32

(52) U.S. Cl. CPC G06Q 40/08; H04L 9/3247; G16H 10/60

(57) ABSTRACT

A cloud-based indemnity medical insurance claiming system and method. The method includes requesting, by a cloud server, claim data such as medical treatment details from a medical institution server when a request to claim indemnity medical insurance is received from a user terminal; extracting, by the medical institution server, patient information and medical treatment information stored in an EMR DB, and mapping, by an API builder, the medical treatment information according to a unique AP; and authenticating, by the medical institution server, the mapped medical treatment information through an electronic signature of a medical institution, encrypting the mapped medical treatment information, and transmitting EDI data including the medical treatment information to the cloud server.

### FIELD

The present disclosure relates to a cloud-based indemnity medical insurance claiming system and method, and more particularly, to a cloud-based indemnity medical insurance claiming system and method for transmitting EDI data including an electronic signature of a medical institute to enable insurance claim processing in a cloud computing environment and transmitting the EDI data to an insurer, so that insurance is claim easily anytime and anywhere.

### BACKGROUND

Indemnity medical insurance is health insurance that guarantees compensation for the actual amount of medical expenses paid by an insured in case of hospitalization or outpatient treatment due to illness or injury. In general, in order to make a claim for insurance, the insured visits a hospital or pharmacy, obtains various types of documentary evidence such as a medical expense bill, a diagnosis, a prescription, and a prescription medicine expense receipt, and then transmits the documentary evidence to the insurer via fax or email.

As described above, in order to claim indemnity medical insurance, it is required to obtain documentary evidence from a medical institution, fill out a claim required by the insurer, and file a claim with the insurer. However, due to the complicated and inconvenient procedure, many insurers do not claim the insurance even though they are entitled to receive insurance premium according to the insurance contract when small medical expenses incur.

In addition, there is a demand for simplification of the medical insurance claim process because issuance of various types of documentary evidence increases unnecessary workload and wasteful use of paper in medical institutions and an input error of claim data can happen in insurers.

As a related art, there is Korean Patent Application Publication No. 10-2016-0069684 (titled FINANCIAL INSTITUTION SERVER AND AUTOMATIC INSURANCE REQUESTING METHOD THEREOF), but this related art merely discloses a technology for claiming insurance premium by photographing a medical expense bill and analyzing an image thereof.

### SUMMARY

An objective of the present disclosure is to provide a system and a method for transmitting EDI data including an electronic signature of a medical institution to a cloud server and transmitting the EDI data to an insurer so that insurance is claimed easily.

A systematic standard procedure for respective processes of individual medical institutions and insurers may be introduced, and there may be provided a cloud-based indemnity medical insurance claiming system and method capable of extracting medical treatment information as EDI data through calculation of a standardized API, regardless of a DBMS and a development language of a corresponding medical institution server, thereby effectively enabling insurance claim in a cloud computing environment.

Another objective of the present disclosure is to simplify a process using a mobile device.

In an existing processing method, there was a procedure in which a user prints and submits documentary evidence to an insurer, and the insurer also inputs claimed data. However, according to an embodiment of the present disclosure, it is possible to transmit electronically-signed original data in the form of EDI or electronic document to a cloud-based system through a mobile terminal of a user, without the existing processes for printing and inputting documentary evidence.

In addition, indemnity insurance claim data transmitted to the cloud may be transmitted to an individual insurer through a specialized communication method and JavaScript Object Notation (JSON) by opening a virtual private network.

Yet another objective of the present disclosure is to accurately claim information issued by a medical institution to an insurer. It is possible to transmit medical treatment information of a person (patient) as it is to an insurer through authentication of electronically-signed original data, authentication of a user terminal, and an app forgery/falsification preventing technology.

In an aspect, a cloud-based indemnity medical insurance claiming method is provided, and the method includes: requesting, by a cloud server, claim data such as medical treatment details from a medical institution server when a request to claim indemnity medical insurance is received from a user terminal; extracting, by the medical institution server, patient information and medical treatment information stored in an EMR DB, and mapping, by an API builder, the medical treatment information according to a unique AP; and authenticating, by the medical institution server, the mapped medical treatment information through an electronic signature of a medical institution, encrypting the mapped medical treatment information, and transmitting EDI data including the medical treatment information to the cloud server.

In another aspect, a cloud-based indemnity medical insurance claiming system is provided, and the system includes: a cloud server configured to request indemnity medical insurance claim from a medical institute server when a request to claim indemnity medical insurance is received from a user, to generate at least one of a receipt, a diagnosis, a prescription, a prescription medicine expense receipt, and a detailed medical expense bill when EDI data including medical treatment information is received from the medical institute server, and to delete the EDI data when insurance premium receipt information is confirmed by a user terminal; the medical institute server configured to extract patient information and medical treatment information from an EMR DB when a request to claim indemnity medical insurance is received from the cloud server, and to map the medical treatment information through an API builder using a unique API, wherein the API builder authenticates the mapped medical treatment information through an electronic signature of a medical institute, encrypts the medical information, and transmits EDI data including the encrypted medical information to the cloud server; and an insurer server configured to calculate insurance premium based on an insurance contract with a patient when the EDI data including the medical information is received from the cloud server, and to request medical treatment details from the could server, wherein the clouds server receives a request to confirm whether a claim is made for unclaimed details by checking the medical information, and the insurer server requests confirmation of personal information regarding the unclaimed details.

According to the present disclosure, it is possible to develop EDI data for insurance claim, regardless of a heterogeneous DBMS of a medical institution server and a type of development language, through a standardized API.

In addition, according to the present disclosure, it is possible to inquire insurance subscription details for each insurer with which a contract is made with an individual using a user terminal.

In addition, according to the present disclosure, it is possible to inquire and make an indemnity medical insurance claim conveniently regardless of time and place using the user terminal, thereby improving user convenience.

In addition, it is possible to increase the processing efficiency through computerization of data input for the indemnity medical insurance claim.

### BRIEF DESCRIPTION OF THE DRAWINGS

FIG. 1 is a flowchart illustrating a cloud-based indemnity medical insurance claiming method according to an embodiment of the present disclosure.

FIG. 2 is a block diagram of a cloud-based indemnity medical insurance claiming system according to an embodiment of the present disclosure.

FIG. 3 is a flowchart illustrating an operation method of an API builder according to an embodiment of the present disclosure.

FIG. 4 is another flowchart illustrating an operation method of an API builder according to an embodiment of the present disclosure.

FIG. 5 is a block diagram of an API builder according to an embodiment of the present disclosure.

### DETAILED DESCRIPTION OF EXEMPLARY EMBODIMENTS

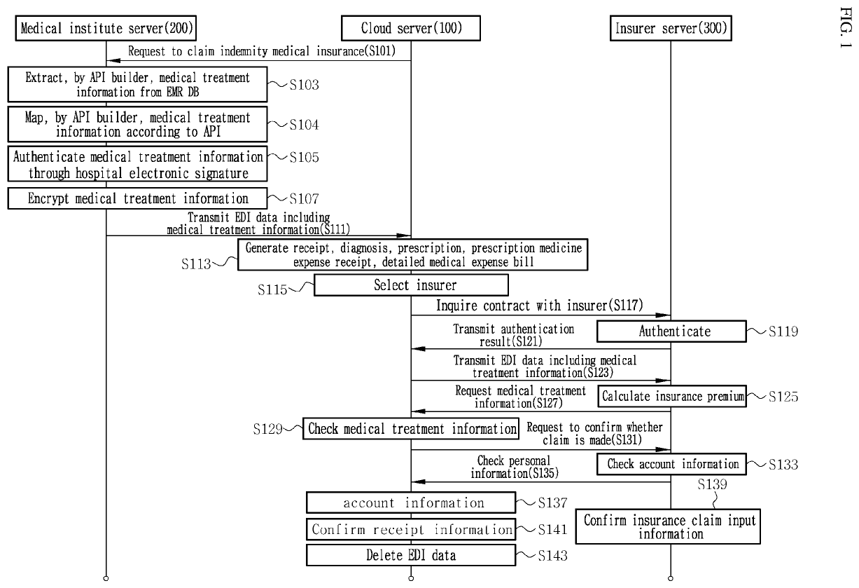

FIG. 1 is a flowchart illustrating a cloud-based indemnity medical insurance claiming method according to an embodiment of the present disclosure.

Referring to FIG. 1, in a cloud-based indemnity medical insurance claiming, when a request for insurance claim for indemnity medical insurance is received from a user terminal 400, a cloud server 100 requests a medical institution server 200 to claim indemnity medical insurance in operation S101.

A cloud-based service may configure a computing environment of virtualization and distributed processing and may be classified as Iaas, Paas, or Saas according to a resource provided, and the present disclosure may configure a platform-based service (Paas) that provides an environment for developing and executing an application.

A secure data input environment is provided through mobile virtual keypad encryption, and mobile security is provided with a Qwerty keypad and a keypad exclusive for English/number/special characters. In addition, by applying SSL, it is possible to strengthen the communication security and apply an in-app data security technology using an encryption algorithm such as AES256.

User authentication may be performed through the ID/PassWord policy of a mobile service, storage of important information in a terminal may be prohibited for confidentiality and integrity of information, and a terminal security policy may be implemented through an app forgery/falsification preventing technology

A medical institution server 200 extracts patient information and medical treatment information stored in an EMR DB 210 in operation S103, and maps the medical treatment information in an application programming interface (API) builder 220 according to an API in operation S104. The medical treatment information may include medicine price information and prescription information. The API is a standardized API that enables mapping into standardized medical treatment information regardless of a heterogeneous Database Management System (DBMS) and a type of development language. The API builder 220 may generate, manage, and test the API for standardizing database information, such as medical treatment information, stored in a medical institution server. The API builder 220 may be installed and operated in the medical institution server, but aspects of the present disclosure are not limited thereto. The API builder 220 generates a data source by extracting the medical treatment information from the EMR DB and maps the medical treatment information by writing a SQL query and repeatedly performing a test in operation S104.

The medical institution server 200 authenticates the mapped medical treatment information through an electronic signature of the medical institution in operation S105, and encrypts the mapped medical treatment information in operation S107. The medical institution server 200 transmits EDI data including medical treatment information to the cloud server 100 in operation S111. The EDI data may include the mapped medical treatment information and the patient information.

The cloud server 100 generates at least one of a receipt, a diagnosis, a prescription, a prescription medicine expense receipt, and a detailed medical expense bill based on the EDI data in operation S113. That is, the cloud server may convert at least one of the receipt, the diagnosis, the prescription, the prescription medicine expense receipt, and the detailed medical expense bill according to an insurer-specific form based on the EDI data.

Then, when an insurer is selected by the user terminal 400 in operation S115, the cloud server 100 inquires with the insurer server 300 whether there is an insurance contract with the insurer in operation S117. The insurer server 300 performs authentication to check whether there is an insurance contract with a corresponding patient, and transmits the authentication result to the cloud server 100 in operation S121.

Thereafter, the cloud server 100 transmits the EDI data including the medical treatment information to the insurer server 300 in operation S123, and the insurer server 300 calculates an insurance premium based on the insurance contract with the corresponding patient based on the EDI data in operation S125.

The insurer server 300 requests medical treatment details from the cloud server in operation S127, and the cloud server checks the medical treatment information and requests to confirm whether a claim is made for the unclaimed details in operation S131.

The insurer server 300 checks account information in operation S133, and requests confirmation of personal information regarding the unclaimed details from the cloud server 100. The cloud server 100 checks the account information in operation S137, and then checks receipt information in operation S141. In addition, the insurer server checks the insurance claim input information S139. Furthermore, the cloud server 100 deletes EDI data in which an indemnity medical insurance claim has been completed.

In addition, a pharmacy server is driven by a medicine program and a Demon program, and when the cloud server transmits prescription information to request medicine preparation, the pharmacy server may receive the prescription information and transmit the received prescription information to the medicine program and the medicine program may calculate a medicine price and transmit the calculated medicine price to the Demon program. The Demon program may transmit the medicine price to the cloud server, request payment, and receive payment completion information.

That is, in the present disclosure, it is possible to claim insurance premium any time and anywhere on the Internet by transmitting EDI data including medical treatment information through the cloud server, and when the insurance claim is completed, the insurance claim data is deleted in the could server, thereby enhancing security.

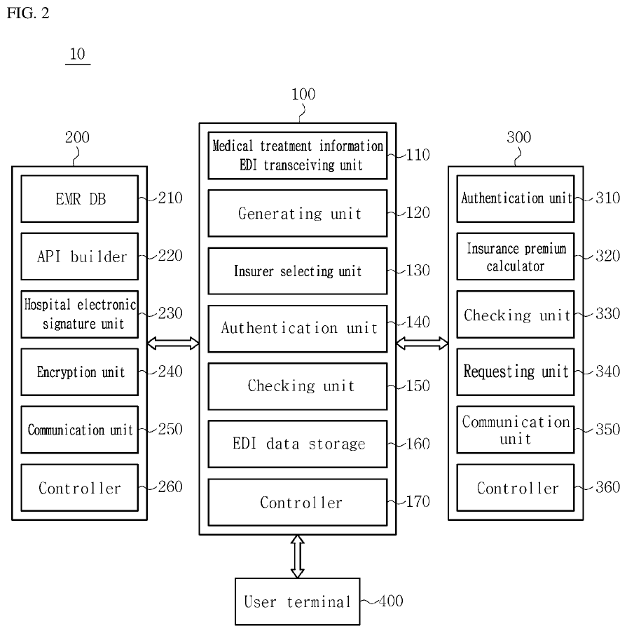

FIG. 2 is a block diagram of a cloud-based indemnity medical insurance claiming system according to an embodiment of the present disclosure. Referring to FIG. 2, a cloud-based indemnity medical insurance claiming system 10 includes a cloud server 100, a medical institution server 200, an insurer server 300, and a user terminal 400. When the user terminal 400 requests indemnity medical insurance, the cloud server 100 requests an indemnity medical insurance claim from the medical institution server 200 and the medical institution server 200 maps and encrypts medical treatment information using a standardized API to provide EDI data including the medical treatment information to the cloud server 100. The cloud server 100 transmits the EDI data to the insurer server 300 to calculate and claim insurance premium.

The cloud server 100 includes a medical treatment information EDI transceiving unit 110, a generating unit 120, an insurer selecting unit 130, an authentication unit 140, a confirmation unit 150, an EDI data storage 160, a controller 170.

When an indemnity medical insurance claim is requested from the user terminal 400, the medical treatment information EDI transceiving unit 110 may claim indemnity medical insurance from the medical institution server 200. In addition, when the EDI data including medical treatment information is received from the medical institution server 200, the medical treatment information EDI transceiving unit 110 may transmit the EDI data to the insurer server 300. The generating unit 120 may generate at least one of a receipt, a diagnosis, a prescription, a prescription medicine expense receipt, and a detailed medical expense bill based on the received EDI data in operation S113.

When an insurer is selected by the user terminal 400, the insurer selecting unit 130 may request to inquire an insurance contract with the selected insurer from a server of the selected insurer.

The authentication unit 140 may perform user authentication upon receiving an authentication result from the insurer server 300. When the medical treatment details or personal information is requested from the insurer server 300, the checking unit 150 may check medical treatment information and account information and transmit the medical treatment information and the account information to the insurer server 300.

When the EDI data is received from the medical institution server 200, the EDI data storage 160 may temporarily store the EDI data, and when insurance premium receipt information is received from the user terminal, the EDI data storage 160 may delete the EDI data stored therein.

The controller 170 may control each configuration of the cloud server.

The medical institution server 200 includes an EMR DB 210, an API builder 220, a medical institution digital signature unit 230, an encryption unit 240, a communication unit 250, and a controller 260.

The EMR DB 210 may store patient information, medical treatment information, prescription information, hospital care information, and history information. The types of information stored in the EMB DB 210 are not limited.

The API builder 220 extracts patient information and medical treatment information from the EMR DB 210 and maps prescription information and the medical treatment information using the API. That is, the API builder generates a data source, and repetitive verification may be performed after simple writing through a SQL query writing guide and mapping to standard data through API builder. In doing so, it is possible to standardize data between heterogeneous DBMSs and data developed in different development languages.

The medical institution electronic signature unit 230 performs authentication on converted medical treatment information through a medical institution electronic signature. The encryption unit 240 may encrypt the mapped medical treatment information to enhance security, the communication unit 250 may transmit and receive data to and from the cloud server 100, and the controller 260 may control each configuration of the medical institution server.

The insurer server 300 includes an authentication unit 310, an insurance premium calculating unit 320, a confirmation unit 330, a request unit 340, a communication unit 350, and a controller 360. When a request to inquire an insurance contract with the insurer is received from the cloud server 100, the authentication unit 310 performs authentication to confirm an insurance contract between a patient and the insurer, and transmits the authentication result to the cloud server 100. The insurance premium calculating unit 320 calculates insurance premium based on the insurance contract with the patient based on the EDI data received from the cloud server.

The check unit 330 may check account information or insurance claim input information.

The requesting unit 340 may request details of medical treatment of the patient from the cloud server, check the medical treatment information in the cloud server 100, and request confirmation of whether a claim is made for unclaimed details.

The communication unit 350 may transmit and receive data to and from the cloud server 100, and the controller 360 may control each configuration of the insurer server.

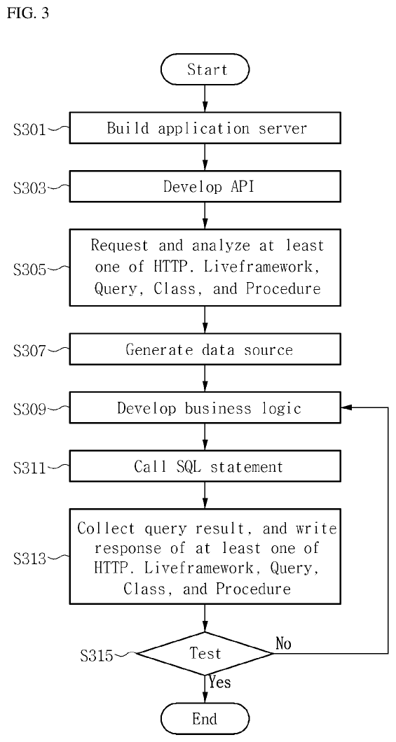

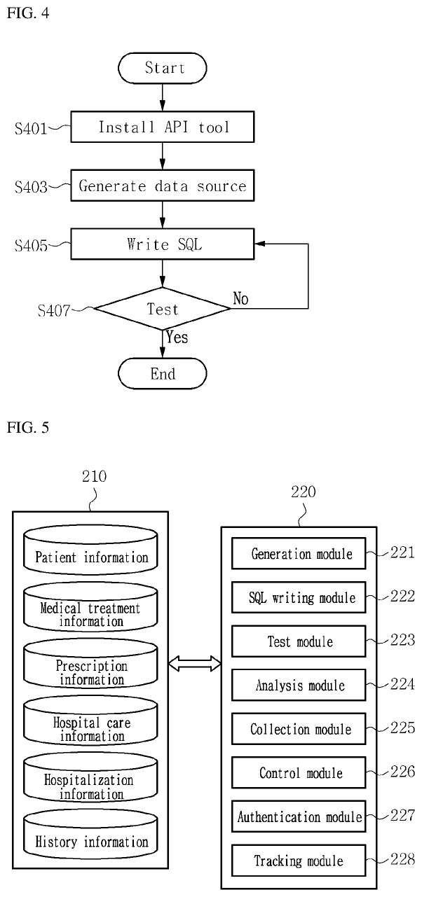

FIGS. 3 and 4 are flowcharts illustrating an operation method of an API builder according to an embodiment of the present disclosure.

Referring to FIGS. 3 and 4, the API builder builds an application server in operation S301. An API is developed in operation S303, and at least one of HTTP, Liveframework, Query Class, and Procedure is requested and analyzed in operation S305. In this case, the application server may be built with IIS, Tomcat, Tuxedo, Entera, etc., but aspects of the present disclosure are not limited thereto. The API may be implemented in .Net, ASP, Java, or C language.

Then, a data source is generated by receiving information from the EMR DB in operation S307, and business logic is developed in operation S309. Then, an SQL statement is called, a query result is collected, and a response of at least one of HTTP Liveframework, Query Class, Procedure, and REST is written in operation S313. If a criterion is met through a test, the procedure ends, or if the criterion is not met, it goes back to the business logic development stage.

Also, in another embodiment, the API builder installs an API tool in operation S401 and generates a data source in operation S403. Then, a SQL query is written in operation S405, and a test is performed repeatedly in operation S407.

FIG. 5 is a block diagram of an API builder according to an embodiment of the present disclosure.

Referring to FIG. 5, the API builder 220 includes a generation module 221, an SQL writing module 222, a test module 223, an analysis module 224, a collection module 225, and a control module 226. The generation module 221 generates a data source by receiving patient information and medical treatment information among the patient information, the medical treatment information, prescription information, hospital care information, and history information stored in the EMR DB 210. The SQL writing module 222 may write an SQL query and transmit the SQL query to the test module 223 to perform a test. The analysis module 224 may analyze an HTTP request. The collection module 225 may collect a query result and write an HTTP response. The control module 226 may control each module of the API builder 220. An authentication module 227 may verify the authenticity of the transmitted data and encrypt the transmitted data. A tracking module 228 may check the insurance claim status of the insurer server in real time after the transmission. That is, it is possible to receive data from the EMR DB 210 and convert the data into an output format standardized by the API builder 220.

While the present disclosure has been described with respect to the specific embodiments, it will be apparent to those skilled in the art that various modifications equivalents may be made from the present disclosure. Therefore, the spirit and scope of the invention should be defined in the appended claims.

1.-11. (canceled)

12. A cloud-based indemnity medical insurance claiming method comprising:
requesting, by a cloud server, claim data such as medical treatment details from a medical institution server when a request to claim indemnity medical insurance is received from a user terminal;
extracting, by the medical institution server, patient information and medical treatment information stored in an EMR DB, and mapping, by an API builder, the medical treatment information according to a unique AP; and
authenticating, by the medical institution server, the mapped medical treatment information through an electronic signature of a medical institution, encrypting the mapped medical treatment information, and transmitting EDI data including the medical treatment information to the cloud server.

13. The cloud-based indemnity medical insurance claiming method of claim 12,
converting, by the cloud server, at least one of a receipt, a diagnosis, a prescription, a prescription medicine expense receipt, and a detailed medical expense bill according to an insurer-specific structure based on the EDI data;
wherein the method further comprises inquiring, by the cloud server, with an insurer server of an insurer selected by a user whether there is an insurance contract made between the user and the insurer.

14. The cloud-based indemnity medical insurance claiming method of claim 13,
further comprising: when an inquiry as to whether there is an insurance contract made between the user and the insurer is received from the cloud server, performing, by the insurer server, authentication based on user information stored in the insurer server and transmitting an authentication result to the cloud server; and
calculating, by the insurer server, insurance premium based on an insurance contract with the user by receiving EDI data including medical treatment information from the cloud server.

15. The cloud-based indemnity medical insurance claiming method of claim 14, further comprising:
requesting, by the insurer server, the medical treatment details from the cloud server, and requesting, by the cloud server, confirmation whether to a claim is made for unclaimed details by checking the medical treatment information; and
checking, by the insurer server, account information and requesting confirmation of personal information regarding the unclaimed details.

16. The cloud-based indemnity medical insurance claiming method of claim 12,
wherein in the converting of the medical treatment information by the API builder, a data source is generated, and repetitive verification is performed after simple writing through a SQL query writing guide and mapping to standard data through API builder

17. A cloud-based indemnity medical insurance claiming system comprising:
a cloud server configured to request indemnity medical insurance claim from a medical institute server when a request to claim indemnity medical insurance is received from a user, to generate at least one of a receipt, a diagnosis, a prescription, a prescription medicine expense receipt, and a detailed medical expense bill when EDI data including medical treatment information is received from the medical institute server, and to delete the EDI data when insurance premium receipt information is confirmed by a user terminal;
the medical institute server configured to extract patient information and medical treatment information from an EMR DB when a request to claim indemnity medical insurance is received from the cloud server, and to map the medical treatment information through an API builder using a unique API, wherein the API builder authenticates the mapped medical treatment information through an electronic signature of a medical institute, encrypts the medical information, and transmits EDI data including the encrypted medical information to the cloud server; and
an insurer server configured to calculate insurance premium based on an insurance contract with a patient when the EDI data including the medical information is received from the cloud server, and to request medical treatment details from the could server, wherein the clouds server receives a request to confirm whether a claim is made for unclaimed details by checking the medical information, and the insurer server requests confirmation of personal information regarding the unclaimed details.

18. The cloud-based indemnity medical insurance claiming method system of claim 17,
wherein the API builder comprises:
a generation module configured to receive the patient information and the medical treatment information from the EMR DB to generate a data source; and
a SQL writing module configured to write a SQL query and transmit the SQL query to a test module.

19. The cloud-based indemnity medical insurance claiming method system of claim 17,
wherein the cloud server comprises an EDI data storage,
wherein when EDI data is received from a hospital server, the EDI data storage temporarily stores the EDI data, and when insurance premium receipt information is received from a user terminal, the EDI data storage deletes the EDI data stored therein.

20. The cloud-based indemnity medical insurance claiming method system of claim 17,
wherein the insurer server comprises:
an insurance premium calculating unit configured to calculate insurance premium based on an insurance contract with a patient based on the EDI data; and
a checking unit configured to check account information and insurance claim input information.

21. The cloud-based indemnity medical insurance claiming method system of claim 20,
wherein the cloud server further comprises a checking unit configured to check the account information or the insurance claim input information.

22. The cloud-based indemnity medical insurance claiming method system of claim 17,
wherein when a request to claim indemnity medical insurance upon completion of medicine payment is received from the user terminal, the cloud server receives EDI data including medical treatment and prescription information from a pharmacy server, and generates at least one of a prescription and a prescription medicine expense receipt, wherein the cloud server deletes the EDI data when insurance premium receipt information is confirmed by the user,
wherein when the EDI data including medical treatment and prescription information is received from the cloud server, the insurer server calculates insurance premium based on an insurance contract with a patient and requests medical treatment and prescription details from the cloud server, wherein the cloud server receives a request to confirm whether a claim is made for unclaimed details by checking the medical treatment and the prescription details and the insurer server requests confirmation of personal information regarding the unclaimed details by checking account information.
